(19) United States
(12) Patent Application Publication
(10) Pub. No.: US 2002/0042932 A1
(43) Pub. Date: Apr. 11, 2002

(54) Process for increasing crop yield or biomass using protoporphyrinogen oxidase gene

(76) Inventors: Kyoung-Whan Back, Kwangju (KR); Hee-Jae Lee, Seoul (KR); Ja-Ock Guh, Kwangju (KR); Sung-Beom Lee, Kwangju (KR)

Correspondence Address: Stephen A. Bent, FOLEY & LARDNER, Washington Harbour, Suite 500, 3000 K Street, N.W., Washington, DC, 20007-5109, US

(21) Appl. No.: 09/877,258

(22) Filed: Jun. 11, 2001

Related U.S. Application Data
(63) Continuation of application No. PCT/KR00/01133, filed on Oct. 10, 2000.

(30) Foreign Application Priority Data
Oct. 11, 1999 (KR) 1999/43860
Nov. 24, 1999 (KR) 1999/52478
Nov. 24, 1999 (KR) 1999/52492

(51) Int. Cl. A01H 5/00; C12N 1/21; C12N 5/06

(52) U.S. Cl. 800/294; 435/252.1; 435/410; 435/320.1

(57) ABSTRACT

This invention relates to a process for increasing crop yield or biomass by enhancing photosynthetic efficiency thereof, which comprises transforming a host crop with a vector containing protoporphyrinogen oxidase (Protox) gene.

### BACKGROUND OF THE INVENTION

The present invention relates to a process for increasing crop yield and biomass using a protoporphyrinogen oxidase (hereinafter, referred to as “Protox”) gene. More specifically, the present invention relates to a process for increasing crop yield and biomass by transforming a host crop with a recombinant vector containing a Protox gene through enhancing photosynthetic capacity of the crop, recombinant vectors, a recombinant vector-host crop system, and use of the recombinant vectors and the recombinant vector-host crop system.

### SUMMARY OF THE INVENTION

Protox which catalyzes oxidation of protoporphyrinogen IX to protoporphyrin IX, is the last common enzyme in the biosynthesis of both heme and chlorophylls. Chlorophylls are light-harvesting pigments in photosynthesis, and thus essential factors associated with photosynthetic capacity and ultimate yield. Thus far, many attempts have been made to increase crop yield through enhancing photosynthetic efficiency; i.e., CO₂ enrichment for increasing photosynthetic capacity [Malano et al., 1994; Jilta et al., 1997], foliar spray of the porphyrin pathway precursor β-aminolevulinic acid for enhancing chlorophyll biosynthesis and thus crop yield [Hotta et al., 1997], and manipulation of gene encoding phytochrome for enhancing photosynthetic efficiency [Clough et al., 1995; Thile et al., Plant Physiol. 1999]. However, these attempts have not been commercialized due to high cost and labor, and possible unexpected side effects that inhibit the crop growth.

To date, a dozen Protox genes have been cloned and characterized from Escherichia coli, yeast, human, and plants, each of which shares low amino acid identities among different organisms, but high homology between closely related families [Dailey et al., 1996; Lermontova et al., 1997; Corrigall et al., 1998].

Although Bacillus subtilis Protox has similar kinetic characteristics to an eukaryotic enzyme which possesses a flavin and employs molecular oxygen as an electron acceptor, it is capable of oxidizing multiple substrates, such as protoporphyrinogen IX and coproporphyrinogen III. Since B. subtilis Protox has lower substrate specificity than eukaryotic Protox, B. subtilis Protox can catalyze the reaction using the substrate for the porphyrin pathway of plants when it is transformed into plants [Dailey et al., 1994].

Protox enzyme has been studied with an emphasis on the weed control and conferring crop selectivity to herbicides [Matringe et al., 1989; Choi et al., 1998; U.S. Pat. No. 5,767,373 (Jun. 16, 1998); U.S. Pat. No. 5,939,602 (Aug. 17, 1999)]. However, no discussion has been made as to Protox in relation to the stimulation of plant growth.

To determine whether the optimal expression of a B. subtilis Protox gene in plant cytosol or plastid stimulates the porphyrin pathway leading to the enhanced biosynthesis of chlorophylls and phytochromes and thereby increasing the photosynthetic capacity of crops, the present inventors developed transgenic rice plants, expressing the B. subtilis Protox gene via Agrobacterium-mediated transformation and examined their growth characteristics in T₀, T₁, and T₂ generations. As a result, they found that the yield and biomass of transgenic rice were considerably increased as a consequence of use of vector-host plant system, and completed the present invention.

Therefore, an object of the present invention is to provide a process for increasing crop yield or biomass by transforming a host crop with a recombinant vector containing a Protox gene, preferably, a B. subtilis Protox gene, through enhancing photosynthetic capacity of the crop. The present invention also includes recombinant vectors, a recombinant vector-host crop system, and use of the recombinant vectors and the recombinant vector-host crop system.

First, the present invention provides a process for increasing crop yield and biomass by transforming a host crop with a recombinant vector containing a Protox gene. In the present process, said gene is preferably a prokaryotic gene and more preferably, a gene from Bacillus or intestinal bacteria. In addition, preferably, said recombinant vector has an ubiquitin promoter and targets to cytosol or plastid of a host plant.

Second, the present invention provides a recombinant vector comprising a Protox gene, an ubiquitin promoter, and a hygromycin phosphotransferase selectable marker. Said Protox gene is preferably isolated from B. subtilis.

Third, the present invention provides A. tumefaciens transformed with the above-described recombinant vector, in particular, an A. tumefaciens LBA4404/pGA1611:C (KCTC 0692BP) or an A. tumefaciens LBA4404/pGA1611:P (KCTC0693BP).

Fourth, the present invention provides a plant cell transformed with the above-described A. tumefaciens. The plant cell may be a monocotyledon; for example, barley, maize, wheat, rye, oat, turfgrass, sugarcane, millet, ryegrass, orchardgrass, and rice or be a dicotyledon; for example, soybean, tobacco, oilseed rape, cotton, and potato.

Fifth, the present invention provides a plant regenerated from the above-described plant cell.

Sixth, the present invention provides a plant seed harvested from the above-described plant.

The development of transgenic plant expressing a B. subtilis Protox gene in T₀, T₁, and T₂ generations will be described hereunder. However, the present invention is not limited to specific plants (e.g., rice, barley, wheat, ryegrass, soybean, potato). One skilled in the art will readily appreciate that the present invention is also applicable to not only other monocotyledonous plants (e.g., maize, rye, oat, turfgrass, sugarcane, millet, orchardgrass, etc.) but also other dicotyledonous plants (e.g., tobacco, oilseed rape, cotton, etc.). Therefore, it should be understood that any transgenic plant using the recombinant vector-host crop system of the present invention lies within the scope of the present invention.

Hereinafter, the present invention will be described in more detail.

Transgenic rice plants expressing a B. subtilis Protox gene via Agrobacterium-mediated transformation are regenerated from hygromycin-resistant callus.

Integration of a B. subtilis Protox gene into plant genome, its expression in cytosol or plastid and inheritance are investigated by using DNA, RNA, Western blots, and other biochemical analyses in T₀, T₁, and T₂ generations of the transgenic rice.

In the present invention, a Protox gene from Bacillus is preferable as a gene source although a Protox gene from an intestinal bacterium such as Escherichia coli can be used. In addition, a recombinant vector having an ubiquitin promoter is preferable. Since B. subtilis Protox has similar substrate specificity to eukaryotic Protox and expression of a gene from a microorganism of which codon usage is considerably different from a plant gene is known to be very low [Cheng et al., 1998], it is believed that the combination of an ubiquitin promoter, a regulatory gene for transgene overexpression in rice, and a B. subtilis Protox gene of which expression is expected to be low in a plant due to its different codon usage from plant gene is favorable for optimal expression of the B. subtilis Protox gene in a plant. If an Arabidopsis Protox gene is expressed in a plastid of a plant using the same recombinant vector as in the present invention, the transgene expression would be much higher compared to the case using a B. subtilis Protox gene or much lower due to the genetic homology of Protox between Arabidopsis and rice. In any case, using the recombinant vector containing a B. subtilis Protox gene is confirmed to produce excellent yields in transgenic rice (see the following table).

Table. Growth characteristics of transgenic rice expressing an Arabidopsis or B. subtilis Protox gene both targeted to a plastid in T₁ generation

| Characteristics | Control | Arabidopsis Protox | B. subtilis Protox |
|---|---|---|---|
| Plant height (cm) | 87 | 75 | 86.5 |
| No. of tillers | 18 | 15 | 35.5 |
| Grain yield (g) | 42.3 | 32 | 69.8 |
| (% of control) | (100) | (75.6) | (165) |

Expression level of a B. subtilis Protox gene in transgenic rice greatly affects grain yield; the transgenic line of C13-1 having higher expression level of a B. subtilis Protox gene was found to have reduced yield increase by 5-10% compared to the transgenic line of C13-2 having optimal expression level of the B. subtilis Protox gene. Therefore, the optimal expression level of the B. subtilis Protox gene is essential for increasing crop yield. Crop yield may be greatly increased by artificial synthesis of the B. subtilis Protox gene introduction of appropriate copy number into a plant genome, and optimal expression of the transgene using various promoters [e.g., cauliflower mosaic virus (CaMV) 35S promoter, rice actin promoter].

Table. Growth characteristics of transgenic rice expressing B. subtilis Protox gene targeted to cytosol according to a promoter in T₁ generation

| Characteristics | Control | Ubiquitin | CaMV 35S | Rice actin |
|---|---|---|---|---|
| Plant height (cm) | 87 | 86.5 | 87 | 84 |
| No. of tillers | 18 | 35.5 | 33 | 32 |
| Grain yield (g) | 42.3 | 69.8 | 65 | 60 |
| (% of control) | (100) | (165) | (153) | (142) |

As shown in the above table, an ubiquitin promoter is the most preferable for expressing B. subtilis Protox gene.

When codon usage of a gene is similar to that of a plant gene (e.g., Protox genes isolated from plants, algae, yeast etc.), however, the optimal expression of these genes is expected to be achieved by using a regulatory gene which is able to control the gene expression.

As the copy number of the introduced B. subtilis Protox gene is increased, its expression level is increased. As the amount of B. subtilis Protox mRNA is increased due to the increased copy number of the transgene, the yield increasing effect is reduced. These observations are set forth in the following table.

Table. Growth characteristics of transgenic rice expressing a B. subtilis Protox gene according to the copy number of the transgene in T₁ generation

| Characteristics | Control | P9 (1 copy) | P21 (3 copies) |
|---|---|---|---|
| Plant height (cm) | 82.5 | 86.5 | 81.5 |
| No. of tillers | 18 | 35.5 | 23.5 |
| Grain yield (g) | 35 | 69.8 | 45.2 |
| (% of control) | (100) | (199) | (129) |

In addition, Western blot analysis of Protox enzyme expressed by a B. subtilis Protox gene in transgenic plants revealed that the transgene expression is higher in the transgenic plants targeting plastid than in those targeting cytosol.

### BRIEF DESCRIPTION OF THE DRAWINGS

FIG. 1 illustrates comparison of the nucleotide sequence (A) and the deduced amino acid sequence (B) of Protox transit peptides (comparison of tobacco Protox sequences of Nicotiana tabacum cv. Samsun and N. tabacum cv. KY16O used in the experiment), and (C) schematic diagram of T-DNA region in binary vector. Ubi, maize ubiquitin; Tnos, nopaline synthase terminator; HPT, hygromycin phosphotransferase; Bs, B. subtilis; Ts, transit sequence.

FIG. 2 illustrates Northern blot analysis of a B. subtilis Protox gene in transgenic rice. C, control; Tc, transgenic control; C8, C13, transgenic rice lines of cytosol targeted; P9, P21, transgenic rice lines of plastid targeted.

FIG. 3 illustrates growth of control and transgenic rice.

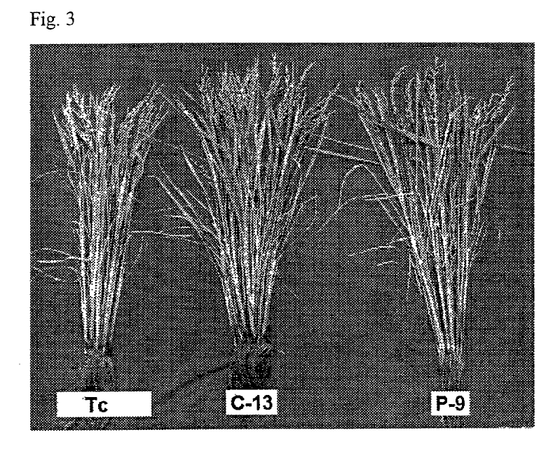

FIG. 4 illustrates DNA (A) and RNA (B) blot analysis of a B. subtilis Protox gene in transgenic rice. C, control; Tc, transgenic control; C8, C13, transgenic rice lines of cytosol targeted; P9, P21, transgenic rice lines of plastid targeted.

### BEST MODE FOR CARRYING OUT THE INVENTION

Specific methods for the present invention are explained hereunder. However, the methods used in the invention and those in the literatures cited can be modified appropriately.

PCR Cloning of the Transit Sequence from Tobacco Protox

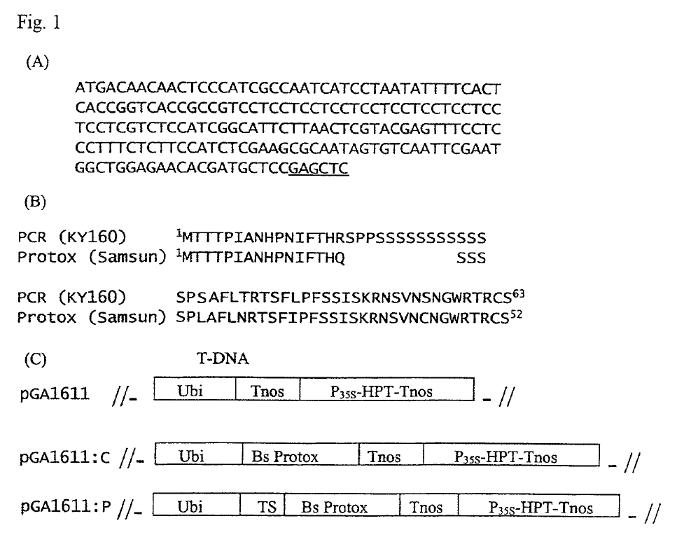

The sequence information of PCR-fished transit sequence showed 189 nucleotides in length with 63 amino acids which has 11 amino acids longer than those of the reported tobacco Protox [Lermontova et al. 1997]. Both deduced amino acid sequences were almost identical except for the 12-consecutive stretch of serine residues in the PCR-fished transit peptide (FIG. 1). However, the sequence variation seemed to be ascribed to the different cultivar of tobacco plants used as a template. The sequence had the common properties of transit peptide such as the richness of Ser/The and the deficiency of Asp/Glu/Tyr [von Heijne et al., 1989].

Transformation Vector Construction

There are numerous binary vectors available for transforming monocotyledonous plants, especially for rice. Almost all of the binary vectors can be obtained from international organizations such as CAMBIA (Center for the Application of Molecular Biology to International Agriculture, GPO Box 3200, Canberra ACT2601, Australia) and university institutes. Transformant selectable marker, promoter, and terminator gene flanked by left or right border region of Ti-plasmid can be widely modified from the basic skeleton of a binary vector.

Although pGA1611 [Kang et al., 1998] as a binary vector is used in Examples of the present invention, other vectors which are capable of a expressing a Protox gene efficiently can be used without any particular limitation. The binary vectors of pCAMBIA 1380 T-DNA and pCAMBIA 1390 T-DNA may be suitable examples, since they have close structural similarity to pGA1611 in the present invention and can be provided by the CAMBIA.

Transformation of Rice

Transformation can be routinely conducted with conventional techniques. Plant transformation can be accomplished by Agrobacterium-mediated transformation and the techniques described in the previous literature [Paszkowsky et al., 1984] can be used. For example, transformation techniques of rice via Agrobacterium-mediated transformation are described in the previous literature [An et al., 1985]. Transformation of monocotyledonous plants can be accomplished by direct gene transfer into protoplasts using PEG or electroporation techniques and particle bombardment into callus tissue. Transformation can be undertaken with a single DNA species or multiple DNA species (i.e., co-transformation). These transformation techniques can be applicable not only to dicotyledonous plants including tobacco, tomato, sunflower, cotton, oilseed rape, soybean, potato, etc. but also to monocotyledonous plants including rice, barley, maize, wheat, rye, oat, turfgrass, millet, sugarcane, ryegrass, orchardgrass, etc. Transformed cells are regenerated into whole plants using standard techniques.

Three gene constructs of pGA1611, pGA1611:C, and pGA1611:P were employed to transform plants using the known molecular biology techniques. These gene constructs were subcloned into a binary vector pGA1611 harboring a constitutive ubiquitin promoter which is known to be appropriately expressed in plants and have hygromycin phosphotransferase as a selectable marker, and transformed into A. tumefaciens LBA4404.

The scutellum-derived calli from rice (Oryza sativa cv. Nakdong) seeds were co-cultivated with A. tumefaciens harboring the above constructs. On average, 10-15% calli were survived from the selection medium containing 50 μg/ml hygromycin. After transferring onto a regeneration medium, selected calli were regenerated into shoots at a rate of 1-5%. During the process of regeneration, some young shoots emerged from the plastid targeted lines (pGA1611:P) were inclined to be etiolated under normal light intensity. However, this phenomenon could be overcome by growing them under dim light condition for 1 week and subsequently transferring them under normal light condition, in which the shoots began to grow normally without being etiolated. It can be explained that these transgenic lines due to the possible overexpression of the B. subtilis Protox gene in the plastid are oxidizing protoporphyrinogen IX into protoporphyrin IX, which is required for the downstream metabolic process, leading to phototoxicity to plant cells (data not shown). On the whole, 6 and 58 different transgenic rice lines having pGA1611:C and pGA1611:P constructs expressed in the cytosol or in the plastid, respectively, were grown to maturity. As a control, a transgenic rice expressing pGA1611 vector was also grown to maturity. Most of the transgenic lines appeared to have normal phenotypes, but their seed production varied ranging from 4 to 260 seeds depending on the individual transgenic lines.

Genomic DNA Gel Blot Analysis

To assess the stable integration of the B. subtilis Protox gene into the rice genome of the transgenic lines regenerated from the hygromycin selection medium, DNA was extracted separately from 5 transgenic lines of cytosol targeted (pGA1611:C) and 6 transgenic lines of plastid targeted (pGA1611:P), digested with HindIII, and hybridized with ³²P-labeled B. subtilis Protox gene. Due to the absence of HindIII site within the probed transgene, the number of hybridized bands directly corresponded to the copy number of the transgene in genome of the transgenic lines. The cytosol targeted transgenic lines (C2, C5, and C6) showed the multiple bands around three hybridizing bands each above 5 kb in size, suggestive of multiple insertions of the transgene at different locations in the rice genome (data not shown). In contrast, lines C8 and C13 had a single copy insertion in the rice genome. As for the plastid targeted transgenic lines, 5 out of 6 plastid targeted transgenic lines had a single copy insertion except the line P21 showing a three-copy insertion (data not shown).

Segregation of Hygromycin-Resistant Trait in Transgenic Rice of T₁ Generation

Seeds from the self-pollinated individual transgenic rice plants of T₀ generation were separately collected for evaluating the segregation of hygromycin-resistant trait in T₁ generation. Five transgenic rice lines including 1 transgenic control (Tc), 2 cytosol targeted lines (C8 and C13), and 2 plastid targeted lines (P9 and P21) were employed in this evaluation. The seeds were germinated on ½ strength MS medium containing 50 μg/ml hygromycin and their survival rates from the medium were recorded for evaluating the segregation of hygromycin-resistant trait. Results are set forth in the following table 1.

Table 1. Segregation of hygromycin-resistant trait in transgenic rice in T₁ generation.

| Transgenic rice | Resistant | Sensitive | Segregation ratio | χ² |
|---|---|---|---|---|
| Tc | 18 | 7 | 3:1 | 0.12 |
| C8 | 19 | 16 | — | — |
| C13 | 22 | 13 | 3:1 | 2.75 |
| P9 | 13 | 7 | 3:1 | 1.07 |
| P21 | 16 | 4 | 3:1 | 0.27 |

Segregation ratios of hygromycin-resistant to sensitive were close to 3:1 in all the transgenic rice lines examined except for line C8, suggesting that the transgene in the rice genome was expressed according to the Mendelian inheritance. In line C8, however, hygromycin-sensitive seeds were found with a high ratio.

RNA Blot Analysis of Transgenic Rice in T₁ Generation

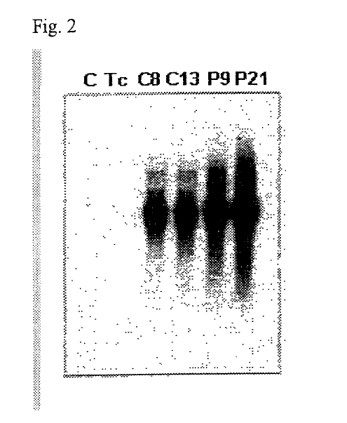

Individuals of transgenic rice lines survived from the medium containing hygromycin (1 transgenic control, Tc; 2 cytosol targeted transgenic lines, C8 and C13; and 2 plastid targeted transgenic lines, P9 and P21) were transplanted into a paddy field. B. subtilis Protox mRNA was not detected in total RNA isolated from the leaves of control (C) and transgenic control (Tc) line (FIG. 2). In the cytosol targeted transgenic lines, C8 and C13 expressed relatively high levels of the B. subtilis Protox mRNA. The plastid targeted transgenic lines were able to transcribe efficiently the B. subtilis Protox gene, in which line P21 exhibited the highest level of the transgene expression.

In view of some relevance between the copy number of transgene and the relative mRNA expression level, the level of the B. subtilis Protox mRNA expression appeared to be associated with the copy number of the transgene in the rice genome. As the copy number of the introduced B. subtilis Protox gene was increased, its expression level was increased (FIG. 2: Transgenic T₁ mRNA blot assay). As the amount of the B. subtilis Protox mRNA was increased due to the increased copy number of the transgene, the yield increasing effect was reduced (see the above table relating to growth characteristics of transgenic rice according to the copy number of the transgene in T₁ generation).

Detection of B. subtilis Protox Polypeptides

Production of B. subtilis Protox protein in transgenic rice of T₁ generation was immunologically examined by using a polyclonal antibody against B. subtilis Protox (source, Rohm and Haas Co.). Soluble proteins were extracted from the leaves of the transgenic rice lines (1 transgenic control, Tc; 2 cytosol targeted transgenic lines, C8 and C13; and 2 plastid targeted transgenic lines, P9 and P21) and electroblotted from gels to PVDF membranes. Subsequent immunodetection of polypeptides on the blot with the antibody against B. subtilis Protox was performed according to standard procedures. Proteins corresponding to B. subtilis Protox in size were detected in all the transgenic rice lines examined except for the transgenic control.

Interestingly, the plastid targeted transgenic lines exhibited 3- to 4-fold higher band intensity than the cytosol targeted lines. Two small protein bands which might be degradation products of B. subtilis Protox were detected in the transgenic lines. In contrast, a faint band larger than B. subtilis Protox by ca. 4-5 kDa was also detected only in the plastid in a targeted transgenic lines. This band was probably proprotein of B. subtilis Protox with non-deleted transit sequence. The antibody-reactive proteins were not detected in microsomal proteins (data not shown).

In conclusion, the detection of degradation products of B. subtilis Protox in the transgenic lines, higher band intensity in the plastid targeted transgenic lines than in the cytosol targeted transgenic lines, and the presence of proprotein of B. subtilis Protox indirectly provide strong evidence for the expression of B. subtilis Protox in the transgenic lines.

DNA and RNA Blot Analysis of Transgenic Rice in T₂ Generation

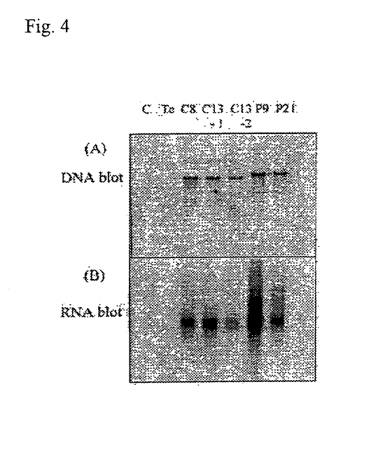

Seeds collected from transgenic rice plants of T₁ generation were germinated and routinely transplanted into a paddy field. Forty plants in each transgenic line were cultivated in the field. At 5 weeks after transplanting, leaves from individual transgenic plants were separately collected to examine the transgene expression according to necrosis response of the leaf segments in distilled water containing 100 mg/l hygromycin. The hygromycin-resistant transgenic lines were analyzed whether the B. subtilis Protox gene was stably expressed in T₂ generation. As in T₁ generation, B. subtilis Protox was found to be expressed in the cytosol targeted transgenic lines (C8 and C13) and in the plastid targeted transgenic lines (P9 and P21) of T₂ generation, but not in control and transgenic control [FIG. 4(A)]. Stable expression of the introduced B. subtilis Protox gene in T₂ generation was confirmed by RNA blot analysis. The levels of B. subtilis Protox mRNA expression were different among the cytosol targeted transgenic lines (C8, C13-1, and C13-2) and between the plastid targeted transgenic lines (P9 and P21) [FIG. 4(B)].

In addition, the transgenic line (FIG. 4, C13-1) having higher expression level of the B. subtilis Protox gene was found to have reduced yield increase by 5-10% compared to the transgenic line (FIG. 4, C 13-2) having the optimal expression level of B. subtilis Protox gene.

The present invention will be specifically explained by reference to the following representative examples. However, these examples are merely illustrative of, and are not intended to limit the present invention in any manner.

### EXAMPLES

### Example 1

### Construction of Transformation Vector for Rice

Two types of B. subtilis Protox gene constructs were used for transforming rice. pGA1611 vector as a starting binary vector was constructed as follows; hygromycin-resistant gene [Gritz and Davies, 1983; NCBI accession No., K01193] as an antibiotic-resistant gene, CaMV 35S promoter [Gardner et al., 1981); Odell et al., 1985; NCBI accession No., V00140] which regulates hygromycin-resistant gene, and termination region of transcription in the 7th transcript of octopine-type TiA6 plasmid [Greve et al., 1982; NCBI accession No., V00088] for terminating transcription were inserted into a cosmid vector pGA482 [An et al., 1988]. Ubiquitin gene [Christensen et al., 1992; NCBI accession No., S94464] was introduced at BamHI/PstI site for expressing foreign gene and the termination region of transcription of nopaline synthase gene [Bevan et al., 1983; NCBI accession No., V00087] was placed at the cloning region having unique restriction enzyme site (HindIII, SacI, HpaI, and KpnI).

A plasmid pGAI6II:C was constructed to express the B. subtilis Protox gene in the cytosol. The full length of polymerase chain reaction (PCR) amplified B. subtilis Protox gene was digested with SacI and KpnI and ligated into pGA1611 binary vector predigested with the same restriction enzymes resulting in placing the Protox gene under the control of the maize ubiquitin promoter. The other construct, pGA1611:P, was designed to target the B. subtilis Protox gene into the plastid (FIG. 1). Sacl primer site designed for the convenient subcloning was underlined. Sequence of tobacco (Nicotiana tabacum cv. Samsun NN) Protox was derived from GenBank database (accession No., Y13465).

For constructing vector, PCR strategy was employed using specific primers which were designed according to the sequence data of tobacco (N. tabacum cv. Samsun NN) Protox. The transit peptide was amplified using the forward primer harboring a HindIII site (underlined) 5′-d(TATCAAGCTTATGACAACAACTCCCATC)-3′, a reverse primer 5′-d(ATTGGAGCTCGGAGCATCGTGTTCTCCA)-3′ harboring a Sacl site (underlined), and tobacco (N. tabacum cv. KY160) genomic DNA as a template. The PCR product was digested with HindIII and Sacl, gel purified, and ligated into the same restriction sites within the pBluescript (commercially available). After verifying the sequence integrity, the HindIII and Sacl fragment of transit sequence was ligated into the same restriction enzyme sites of pGA1611:C vector leading to the construction of pGA1611:P which had placed transit peptide in front of the B. subtilis Protox gene. FIG. 1 illustrates schematic diagram of T-DNA region in binary vector. The abbreviations used in FIG. 1 are as follows; Ubi, maize ubiquitin; Tnos, nopaline synthase 3′ termination signal; P_{35S}, CaMV 35S promoter; HPT, hygromycin phosphotransferase; Ts, transit sequence.

### Example 2

### Transformation and Regeneration of Rice

A. tumefaciens LBA4404 harboring pGA1611, pGA1611:C, and pGA1611:P was grown overnight at 28° C. in YEP medium (1% Bacto-peptone, 1% Bacto-yeast extract, 0.5% NaCl) supplemented with 5 μg/ml tetracyclin and 40 μg/ml hygromycin. The cultures were spun down and pellets were resuspended in an equal volume of AA medium [Hiei et al., 1997] containing 100 μM acetosyringone. The calli were induced from scutellum of rice (cv. Nakdong) seeds on N6 medium [Rashid et al., 1996; Hiei et al., 1997]. The compact calli of 3- to 4-week-old were soaked in the bacterial suspension for 3 minutes, blotted dry with sterile filter paper to remove excess bacteria. The calli were transferred to a co-culture medium and then cultured for 2-3 days in darkness at 25° C. The co-cultured calli were washed with sterile distilled water containing 250 mg/l cefotaxime. The calli were transferred to N6 medium containing 250 mg/l cefotaxime and 50 mg/l hygromycin. After selection for 3-4 weeks, the calli were transferred to a regeneration medium for shoot and root development. After the roots had sufficiently developed, the transgenic plants were transferred to a greenhouse and grown to maturity.

A. tumefaciens transformed with pGA1611:C and pGA1611:P vectors in the present invention have been deposited in an International Depository Authority under the Budapest Treaty (Korean Collection for Type Cultures, Korea Research Institute of Bioscience and Biotechnology, 52 Auheun-dong, Yusung-ku, Taejon 305-333, Korea) on Nov. 15, 1999 as KCTC 0692BP and KCTC 0693BP, respectively.

### Example 3

### Transformation and Regeneration of Soybean

A. tumefaciens LBA4404 harboring pGA1611, pGA1611:C, and pGA1611:P were grown overnight at 28° C. in YEP medium (1% Bacto-peptone, 1% Bacto-yeast extract, 0.5% NaCl) supplemented with 5 μg/ml tetracyclin and 40 μg/ml hygromycin. The cultures were spun down and pellets were resuspended in an equal volume of B5 medium [Gamborg et al. 1968] containing 100 μM acetosyringone. Cotyledon tissues which were longitudinally wounded were co-cultured with the bacterial suspension for 3 days at 24° C. The co-cultured calli were transferred to B5 recovery medium and a regeneration medium [Di et al., 1996] for the generation of T₀ soybean.

### Example 4

### Construction of Transformation Vector for Barley, Wheat, Ryegrass, and Potato

From pGA1611:C and pGA1611:P binary vectors, the genes including ubiquitin promoter, B. subtilis Protox gene, and 3′ termination region of nopaline synthase gene were digested with BamHI/ClaI and ligated into the same restriction enzyme site within pBluscript II SK cloning vector (Strategene, USA) leading to the construction of PBSK-Protox vectors. Region of CaMV 35S promoter:hygromycin-resistant gene:termination region of transcription in octopine-type TiA6 plasmid was digested from pGA1611:C with ClaI/SalI and ligated within pBSK-Protox vector leading to the construction of pBSK-Protox/hygromycin vector as a vector for transformation using a gene gun.

### Example 5

### Transformation and Regeneration of Barley, Wheat, Ryegrass, and Potato

Scutellum-derived calli were used as explants for the transformation of barley, wheat, and ryegrass [Spangenberg et al., 1995; Koprek et al., 1996; Takumi and Shimada, 1997], whereas cotyledon tissues were used for the transformation of potato. The pBSK-Protox/hygromycin vector DNAs coated with 1.6-μm diameter gold particles were bombarded into the explants of barley, wheat, ryegrass, and potato by using a biolistic PDS-1000/He Particle Delivery System (Bio-Rad). B. subtilis Protox protein from the transformed plants was extracted in 1 ml of homogenization medium consisting of 0.1 M Tris buffer (pH 7.0), 5 mM β-mercaptoethanol, and 1 tablet/10 ml of complete protease inhibitors [Complete Mini; Boehringer Mannheim] at 4° C. The homogenate was filtered through 2 layers of Miracloth (CalBiochem) and centrifuged at 3,000 g for 10 minutes. The resulting supernatant was centrifuged at 100,000 g for 60 minutes to obtain crude microsomal pellet. The pellet was resuspended in 100 μl of the homogenization buffer. The resuspended pellet of 20 μg protein was used for immunoblotting against microsomal fraction, whereas the 100,000 g supernatant of 15 μg protein was used as soluble protein. Both soluble and microsomal proteins were subjected to sodium dodecyl sulfate-polyacrylamide gel electrophoresis (SDS-PAGE) using 10% (w/v) acrylamide/bis gel. Following the electrophoresis, the proteins were blotted to PVDF membranes and subsequently immunodetected with a polyclonal antibody against B. subtilis Protox. The application of secondary antibody and band detection was performed using an enhanced chemiluminescence system according to the manufacturer's protocol (ECL Kit; Boehringer Mannheim).

Test 1: Growth Results of Transgenic Rice

Seeds from transgenic rice plants which were regenerated in Example 2 were collected and the hygromycin-resistant seedlings were transplanted into a paddy field. The growth results of the transgenic rice are shown in Tables 2 to 5. Table 2 shows the plant height of the transgenic rice in T₁ generation at different growth stages.

Table 2. Plant height of transgenic rice in T₁ generation at different growth stages.

| Weeks after | Plant height (cm) (average of at least 4 plants) | | | | | |
|---|---|---|---|---|---|---|
| Transplanting | Control | TC | C8 | C13 | P9 | P21 |
| 1 | 26.0 | 28.3 | 28.2 | 25.5 | 25.3 | 26.6 |
| 2 | 43.2 | 41.7 | 40.3 | 45.3 | 43.0 | 41.4 |
| 3 | 46.7 | 46.3 | 45.3 | 48.5 | 43.3 | 47.6 |
| 4 | 53.0 | 52.3 | 49.7 | 51.3 | 48.3 | 55.8 |
| 10 | 82.3 | 79.0 | 86.3 | 89.5 | 85.8 | 79.6 |
| 16 | 82.5 | 79.0 | 86.5 | 90.5 | 86.5 | 81.5 |

As shown in Table 2, the cytosol targeted transgenic rice exhibited significantly higher plant height by 10 cm compared to control.

Tables 3, 4 and 5 show number of tillers, quantitative characteristics, and yield components of transgenic rice in T1 generation, respectively.

Table 3. Number of tillers of transgenic rice in T1 generation at different growth stages (Numbers in parenthesis are percentage relative to control)

| Weeks after | No. of tillers (average of at least 4 plants) | | | | | |
|---|---|---|---|---|---|---|
| transplanting | Control | TC | C8 | C13 | P9 | P21 |
| 1 | 3.6 | 3.7 | 3.3 | 2.8 | 2.3 | 4.2 |
| 2 | 6.3 | 6.0 | 6.0 | 7.5 | 8.0 | 6.8 |
| 3 | 8.8 | 9.3 | 10.3 | 16.0 | 14.3 | 13.6 |
| 4 | 15.7 | 15.7 | 18.7 | 24.3 | 26.3 | 18.7 |
| 10 | 15.7 | 16.2 | 19.3 | 26.5 | 26.3 | 19.5 |
| 16 | 18.0 | 18.2 | 23.0 | 28.0 | 35.5 | 23.5 |
| | (100) | (101) | (128) | (156) | (197) | (131) |

Table 4. Quantitative characteristics of transgenic rice in T₁ generation

| | Con- | | | | | |
|---|---|---|---|---|---|---|
| Characteristics | trol | TC | C8 | C13 | P9 | P21 |
| Shoot fresh weight | 131 | 138 | 246 | 252 | 188 | 171 |
| (g) | | | | | | |
| Root fresh weight | 89 | 92 | 140 | 111 | 93 | 68 |
| (g) | | | | | | |
| Shoot/root fresh | 1.5 | 1.5 | 1.75 | 2.27 | 2.02 | 2.51 |
| weight ratio | | | | | | |
| Panicle length (cm) | 20.2 | 18.7 | 17.3 | 19.1 | 19.6 | 18.3 |
| Effective tillering | 82.1 | 76.9 | 89.1 | 93.9 | 80.9 | 77.9 |
| ratio | | | | | | |

Table 5. Yield components of transgenic rice in T₁ generation

| Yield components | Control | TC | C8 | Cl3 | P9 | P21 |
|---|---|---|---|---|---|---|
| Grain yield (g) | 35.0 | 35.2 | 36.3 | 58.6 | 69.8 | 45.2 |
| (% of control) | (100) | (101) | (104) | (167) | (199) | (129) |
| 1,000 grain weight (g) | 28.3 | 30.0 | 27.7 | 31.4 | 29.2 | 28.2 |
| No. of panicles | 15.0 | 14.0 | 20.5 | 26.3 | 28.7 | 18.3 |
| No. of grains per | 94.4 | 94.0 | 99.4 | 108 | 104 | 101 |
| panicle | | | | | | |
| Grain filling ratio (%) | 88.1 | 85.5 | 85.9 | 84.8 | 86.0 | 86.7 |

As shown in Tables 3, 4 and 5, the quantitative characteristics, i.e., effective tillering ratio was significantly improved in the transgenic rice by the present invention and their grain yield and number of tillers were also increased as much as 2 times.

Test 2: Growth Results of Transgenic Barley, Wheat, Soybean, Italian Ryegrass, and Potato

The growth characteristics of the transgenic monocotyledonous plants (barley, wheat), dicotyledonous plants (soybean, potato), and forage crop (Italian ryegrass) which were all regenerated similarly as in Example 2 were examined. Grain yield increase by 18-27% was observed in the transgenic barley (Table 6). Grain yield increases by 14-25% and 23-28% were observed in the transgenic wheat (Table 7) and soybean (Table 8), respectively. In the case of the transgenic Italian ryegrass, shoot fresh weight was increased by up to 51% (Table 9). Table 10 shows yield characteristics of transgenic potato. Both shoot and tuber fresh weights were increased by 13-18%. These results demonstrate that yield increase effect by B. subtilis Protox gene can be widely applicable not only to monocotyledonous plants including rice but also to forage crops and dicotyledonous plants.

Table 6. Yield characteristics of transgenic barley

| Characteristics | Control | TC | C112 | P115 |
|---|---|---|---|---|
| Grain yield (g) | 177 | 180 | 228 | 211 |
| (% of control) | (100) | (100) | (127) | (118) |
| 1,000 grain weight (g) | 34.9 | 33.8 | 33.1 | 31.4 |
| No. of panicles | 4.3 | 4.0 | 6.3 | 5.5 |
| No. of grains per panicle | 42.0 | 44.2 | 51.4 | 47.0 |
| Grain filling ratio (%) | 82.7 | 82.0 | 80.1 | 84.5 |
| Panicle length (cm) | 3.9 | 3.8 | 4.0 | 4.2 |
| Plant height (cm) | 69.5 | 67.4 | 69.0 | 70.8 |

Table 7. Yield characteristics of transgenic wheat

| Characteristics | Control | TC | C204 | P207 |
|---|---|---|---|---|
| Grain yield (g) | 247 | 242 | 310 | 282 |
| (% of control) | (100) | (97) | (125) | (114) |
| 1,000 grain weight (g) | 45.3 | 44.0 | 46.1 | 45.0 |
| No. of panicles | 5.6 | 5.3 | 7.2 | 8.3 |
| No. of grains per panicle | 34.2 | 36.0 | 40.1 | 37.0 |
| Grain filling ratio (%) | 80.6 | 79.2 | 77.1 | 81.0 |
| Panicle length (cm) | 7.8 | 7.1 | 7.6 | 7.7 |
| Plant height (cm) | 67.4 | 69.0 | 76.4 | 72.0 |

TABLE 8
Yield characteristics of transgenic soybean

| Characteristics | Control | TC | C303 | P310 |
|---|---|---|---|---|
| Grain yield (g) | 39.2 | 36.5 | 48.5 | 50.3 |
| (% of control) | (100) | (94) | (123) | (128) |
| 1000 grain weight (g) | 19.6 | 21.0 | 20.0 | 22.5 |
| Plant height (cm) | 71.4 | 68.4 | 78.0 | 76.3 |
| Grain filling ratio (%) | 80.2 | 81.0 | 90.4 | 87.4 |

TABLE 9
Yield characteristics of transgenic Italian ryegrass

| Characteristics | Control | TC | P407 |
|---|---|---|---|
| Shoot fresh weight (g) | 117 | 105 | 178 |
| (% of control) | (100) | (89) | (151) |
| No. of tillers | 8.5 | 8.0 | 12.3 |
| No. of leaves | 36.0 | 41.2 | 50.0 |

TABLE 10
Yield characteristics of transgenic potato

| Characteristics | Control | TC | C401 | P421 |
|---|---|---|---|---|
| Shoot fresh weight (g) | 55 | 52 | 62 | 65 |
| (% of control) | (100) | (95) | (113) | (118) |
| Plant height (cm) | 85 | 82 | 80 | 78 |
| Tuber fresh weight (g) | 135 | 130 | 155 | 160 |

### Industrial Applicability

Since significant increases in crop yield and biomass by transforming a host crop with a recombinant vector containing a Protox gene according to the present invention are confirmed, food shortage problem can be solved and the enhanced utilization of plant resources including forage crops can be secured with the present invention.

What is claimed is:

1. A process for increasing crop yield and biomass by transforming a host plant with a recombinant vector containing a protoporphyrinogen oxidase (Protox) gene.

2. The process of claim 1 wherein said gene is a prokaryotic gene.

3. The process of claim 2 wherein said prokaryotic gene is derived from a Bacillus or intestinal bacteria.

4. The process of claim 1 wherein said recombinant vector has an ubiquitin promoter.

5. The process of claim 1 wherein said recombinant vector targets cytosol or plastid of the host plant.

6. A recombinant vector comprising a protoporphyrinogen oxidase (Protox) gene, an ubiquitin promoter, and a hygromycin phosphotransferase selectable marker.

7. The recombinant vector of claim 6 wherein said protoporphyrinogen oxidase (Protox) is derived from Bacillus subtilis.

8. An Agrobacterium tumefaciens transformed with the recombinant vector of claim 6.

9. The Agrobacterium tumefaciens of claim 8 which is an Agrobacterium tumefaciens LBA4404/pGA1611:C (KCTC 0692BP) or an Agrobacterium tumefaciens LBA4404/pGA1611:P (KCTC0693BP).

10. A plant cell transformed with the Agrobacterium tumefaciens of claim 8 or claim 9.

11. The plant cell of claim 10 wherein said plant is a monocotyledon.

12. The plant cell of claim 11 wherein said monocotyledon is selected from the group consisting of barley, maize, wheat, rye, oat, turfgrass, sugarcane, millet, ryegrass, orchardgrass and rice.

13. The plant cell of claim 10 wherein said plant is a dicotyledon.

14. The plant cell of claim 13 wherein said dicotyledon is selected from the group consisting of soybean, tobacco, oilseed rape, cotton and potato.

15. A plant regenerated from the plant cell of claim 10.

16. A plant seed harvested from the plant of claim 15.
